(19) United States
(12) Patent Application Publication
(10) Pub. No.: US 2010/0291604 A1
(43) Pub. Date: Nov. 18, 2010

(54) PREDICTIVE TREATMENT OF DYSGLYCEMIC EXCURSIONS ASSOCIATED WITH DIABETES MELLITUS

(76) Inventors: Paul M. Rosman, Lyndhurst, OH (US); Scott M. Pappada, Warren, OH (US)

Correspondence Address: HOWARD M COHN;PATENT ATTORNEY LLC, 21625 CHAGRIN BLVD #220, CLEVELAND, OH, 44122, US

(21) Appl. No.: 12/770,378

(22) Filed: Apr. 29, 2010

Related U.S. Application Data
(63) Continuation of application No. 11/615,212, filed on Dec. 22, 2006.

(30) Foreign Application Priority Data
Jun. 28, 2004 (US) PCT/US2004/020643

(51) Int. Cl. C12Q 1/54; C12M 1/34

(52) U.S. Cl. 435/14; 435/287.1

(57) ABSTRACT

A predictive technique for treating diabetes mellitus is described whereby a patient's blood glucose levels are monitored “continuously” over an extended period of time and a life-event diary is maintained records all significant life-events (e.g., food intake, medication, exercise, mood/emotions, etc.). This information is analyzed to derive a mathematical model that closely matches the patient's glucose level variations for the period of monitoring. Specific daily time periods of dysglycemic vulnerability are determined by calculating when the mathematical model predicts that crossings of predetermined hyperglycemic and hypoglycemic threshold levels will occur. These predicted periods of vulnerability are then used to devise a therapeutic plan that administers treatment in anticipation of predicted dysglycemic excursions, thereby limiting the extent of those excursions or eliminating them altogether.

### CROSS-REFERENCE TO RELATED APPLICATIONS

This application is a continuation of copending U.S. application Ser. No. 11/615,212, filed on Dec. 22, 2006, incorporated herein by reference, and which in turn claims the benefit of PCT Patent Application No. PCT/US2004/020643, filed on Jun. 28, 2004.

### TECHNICAL FIELD OF THE INVENTION

The present invention relates to the treatment of diabetes mellitus, and more particularly to the treatment of dysglycemic excursions associated with diabetes mellitus.

### BACKGROUND

Diabetes mellitus is a name used to refer to a group of metabolic diseases characterized by high blood sugar (glucose) levels resulting from defects in insulin production, insulin action or a combination of the two. In normal individuals (i.e., in individuals free of the disease), a natural body mechanism associated with the pancreas controls blood glucose levels tightly by releasing insulin in response to increases in blood glucose levels. Insulin acts to reduce blood glucose levels. In patients with diabetes mellitus, however, insufficient production and/or inaction of insulin causes hyperglycemia.

Diabetes mellitus has been known since ancient times. Commonly referred to simply as “diabetes”, diabetes mellitus means “sweet urine.” This name derives from the fact that in individuals with the disease, elevated levels of blood glucose (hyperglycemia) lead to the excretion of glucose into the urine. The ancient Hindus were the first to coin the term “honey urine,” a thousand years before the first Europeans recognized the sweet taste of urine in patients with diabetes. They accurately described polyuria and glycosuria, noting the attraction of flies and ants to the urine of those affected by this ailment.

In 1865, Claude Bernard determined that “something” controlled glucose levels in the blood and that diabetes mellitus occurred because that “something” was deficient or missing. In 1922, Banting, Best and McCollough identified insulin as that “something” in the pancreas of a dog. By 1929, Joslin and colleagues purified insulin from animal pancreatic extracts sufficiently that it could be administered to humans, thereby making it possible to survive diabetes mellitus with proper treatment.

Diabetes mellitus is a chronic disease that requires long-term medical attention both to limit the development of its devastating complications and to manage them when they do occur. Compared to other disorders, diabetes is a disproportionately expensive disease. In 1997, patients diagnosed with diabetes accounted for 5.8% of the US population, or 15.7 million people, but their per capita health care cost was $10,071, as compared to $2,699 for those without diabetes. During this same year, diabetes accounted for 30.3 million physician office visits and 13.9 million days of hospital stay. Diabetes is the third leading cause of death in the United States after heart disease and cancer.

Long-term complications of diabetes include problems involving the eyes, kidneys and nerves, all generally a result of poor blood flow due to diabetes-related damage to small blood vessels. The primary eye complication related to diabetes is diabetic retinopathy, resulting by retinal scarring and/or retinal detachment, ultimately leading to impaired vision or blindness. Kidney damage from diabetes is known as diabetic nephropathy, resulting in impaired kidney function or complete kidney failure. Nerve damage from diabetes is known as diabetic neuropathy, wherein poor blood flow to the nerves causes nerve damage or destruction, especially those in the lower extremities. This produces symptoms such as numbness, burning and aching of the feet and lower extremities. Compounded by poor blood circulation, this can lead to foot injuries that do not heal, often leading to serious infection, ulcers and even gangrene, necessitating amputation of the affected parts.

Since the first production of insulin for human use by Joslin et al. in 1929, insulin has been characterized on a molecular level and the physiology of glucose-insulin action has been defined. This characterization of molecular and physiologic aspects of human glucose control includes the timing of glucose excursions and of the changes in the relationships between insulin action and glucose responsiveness during the 24-hour day.

As the pathophysiology of diabetes mellitus became better understood, technologies were developed to allow for simple, convenient measurement of blood glucose levels throughout the day in diabetic individuals. Most modern clinical techniques for glucose measurement are primarily episodic, involving discrete, relatively infrequent measurements of blood glucose by “finger-stick”, performed on the patient either by the patient himself/herself or by others. Recently, however, “continuous” glucose measurement has been FDA approved and marketed for clinical use. One example of this is the Continuous Glucose Monitoring System (CGMS®) developed by Medtronic MiniMed Corporation, which measures glucose levels every 5 minutes for 3 days, and reports the information after the 3-day period is completed. Others have developed methods of reporting glucose levels concurrently with measurement. The clinical usefulness of the concurrent reporting feature, however, appears to be limited.

Avoiding low and high glucose levels (dysglycemia) is vital to the clinical management of diabetes mellitus, and in many patients current approaches are unsuccessful as measured by the occurrence of acute and chronic complication, and by the immense cost of diabetes care in the country and others.

Low blood glucose levels are potentially devastating because they can produce coma and lesser degrees of brain dysfunction that can result in injury or death. Low blood glucose levels may be unrecognized by people who have diabetes mellitus for several years, thereby generating an added danger. Furthermore, low blood glucose levels are the major impediment to clinically acceptable glucose level control in insulin dependent diabetes mellitus patients.

High blood glucose levels are associated with increased risk of devastating long term complications in all people with diabetes mellitus. These complications include microvascular and macrovascular problems. Microvascular complications of diabetes mellitus include retinopathy (and visual loss), Nephropathy (and renal failure) and neuropathy (and loss of feeling, altered sensation, severe pain, or inability to recognize low blood glucose levels). Macrovascular complications of diabetes mellitus include myocardial infarction, increased cardiac death, and stroke. All of these complications are reduced by improved blood glucose control and many are reversible over time if glucose levels are normalized.

Current diabetes treatment regimens, based upon episodic patient obtained finger stick glucose measurements, are proving to be inadequate to obtain clinical diabetes management targets because blood glucose levels can fall by 50% in 20 minutes, or increase by 200% in 15 minutes, depending upon the circumstance. Further, significant changes in glucose levels occur when these patients are sleeping.

### SUMMARY OF THE INVENTION

The present inventive technique provides a predictive technique for treating diabetes mellitus wherein a patient's blood glucose levels are monitored “continuously” (recorded repeatedly over very short intervals, e.g., every 5 minutes) over an extended period of time, e.g., 72 hours or more. A life-event diary is maintained during monitoring to record all significant life-events (e.g., food intake, medication, exercise, mood/emotions, etc.). This information is then analyzed to derive a mathematical model that closely matches the patient's glucose level variations for the period of monitoring. Specific daily time periods of dysglycemic vulnerability are determined by calculating when the mathematical model predicts that crossings of predetermined hyperglycemic and hypoglycemic threshold levels will occur.

These predicted periods of vulnerability are then used to devise a therapeutic plan that administers treatment in anticipation of predicted dysglycemic excursions, thereby limiting the extent of those excursions or eliminating them altogether.

According to the invention, a patient's blood glucose levels arc monitored and recorded continuously over an extended period of time. During the period of monitoring, the patient records relevant life-event information into a life-event dairy. Recorded information includes all life-events of significant relevance to glucose level fluctuations, such as emotional state, level of activity/exertion, food intake, insulin dosages, etc. At the end of the monitored period, the recorded glucose level and life-event information is analyzed to identify correlations between specific life events and periodicities in monitored blood glucose level variations. From this analysis, a predictive sinusoidal function is determined that “models” the patient's daily glycemic pattern (over the monitored period).

From the model sinusoidal function, daily times can be determined (predicted) when the patient's blood glucose levels are expected to cross predetermined hypoglycemic and hyperglycemic threshold crossings, based upon times when the sinusoidal function crosses those threshold levels.

According to an aspect of the invention, periods of time between threshold crossings define time periods (windows) of vulnerability during which the patient is ordinarily expected to experience dysglycemic excursions. Based upon these threshold crossing times and the associated periods of vulnerability, a plan of treatment can be developed such that treatment (e.g., insulin or glucose) can be administered in anticipation of an expected dysglycemic event. Preferably, this treatment will either minimize the depth and duration of the dysglycemic excursion or prevent it altogether.

According to another aspect of the invention, life-event information is correlated with recorded glucose level information to identify causal (or apparently causal) relationships between specific life events (e.g., intensive exercise) and corresponding glucose level fluctuations.

According to another aspect of the invention, the model sinusoidal function is determined by Fourier analysis of the recorded glucose level information to identify the phase and amplitude of circadian/ultradian Fourier (sinusoidal) components corresponding to glucose level variations. These sinusoidal components are then used to provide a model of the patient's daily glucose level patterns, thereby providing a basis for prediction of dysglycemic events.

According to another aspect of the invention, a computing device is used to record life-event data for a patient. The patient is provided with the computing device, which can be a personal computer, a PDA (personal digital assistant) or dedicated life-event “calculator” (essentially a fixed-function computing device for recording life-event data). During the period of continuous glucose monitoring, the patient records life-event information in electronic, computer-readable form via the computing device.

### BRIEF DESCRIPTION OF THE DRAWINGS

These and further features of the present invention will be apparent with reference to the following description and drawing, wherein:

FIG. 1 is a block diagram showing the various elements of the present inventive technique.

FIG. 2 is an illustration of a Life Event Diary form illustrating the type and organization of data to be collected from a patient, in accordance with the present invention.

FIG. 3 is a table illustrating quantization of activity levels and emotional states, in accordance with the invention.

FIGS. 4A and 4B are graphs of continuous glucose monitoring data on two consecutive days for a representative diabetic patient. in accordance with the invention

FIG. 5 is a table showing the organization of data into relevant time periods, according to the invention.

FIG. 6 is a graph of continuous glucose monitoring data, in accordance with the invention.

FIG. 7 is a graph of the continuous glucose monitoring data of FIG. 7 after smoothing, in accordance with the invention.

FIG. 8 is a graph showing a sine curve fit to the smoothed continuous glucose monitoring data of FIG. 7, in accordance with the invention.

FIGS. 9 and 10 are tables showing the relationship between predicted times of dysglycemic excursions and specific glucose sensor readings on specific days of monitoring, in accordance with the invention.

### DETAILED DESCRIPTION OF THE INVENTION

The ultimate goal of research into the treatment of diabetes mellitus is the complete cure and elimination of the disease. Some of this research is directed towards transplantation of insulin producing pancreatic islets, and towards whole pancreas organ transplantation. As with many types of transplantation, the practicality of pancreatic transplantation is severely limited by the general lack of availability of organs and resources. Other research is directed towards the production of an artificial pancreas capable of measuring blood glucose levels and immediately adjusting insulin infusions to avoid low and high glucose excursions. However, this research has not yet produced results sufficient to provide practical treatment of diabetes mellitus on a large scale.

Present treatment strategies for regulating glucose levels by administering insulin are generally of a reactive nature. That is, they are responsive to measurements of blood glucose levels that have already occurred. By their very nature, therefore, reactive treatment techniques will always lag the onset of dysglycemic excursions by some period of time, even when blood glucose levels arc monitored continuously. Accordingly, reactive treatment regimens can only respond to dysglycemic events when they are already in progress, greatly limiting their effectiveness against large and sudden dsyglycemic excursions.

By way of contrast, the present inventive technique is directed toward the anticipation and prevention of dysglycemic excursions by predicting dysglycemic events and administering appropriate treatment as blood glucose levels are about to change. One of the greatest advantages of this technique is that it allows treatment to get ahead of large dysglycemic excursions in blood glucose levels, ideally preventing those excursions from ever occurring.

The present inventive technique identifies and analyzes recurrent blood glucose level patterns in individual patients as a platform for effective, personalized diabetes management. By continually monitoring blood glucose levels over an extended period of time (e.g., several days at least) and by recording and taking into account certain relevant life events (e.g., food intake (type and amount), insulin administration (type and dose), level of activity, mood, sleep patterns, exercise etc.) it develops a predictive glucose-insulin model (algorithm) by which blood glucose level variations can be anticipated and treated. Since insulin sensitivity may be different on different days, there is a need to develop separate glucose-insulin algorithms for specific situations. Time domain analysis is one approach by which algorithm adjustment can be accomplished for diabetic patients with varying insulin requirements.

Hypoglycemia is generally considered to be the most important limiting factor in effective diabetes management in many patients. One of the significant motivations for the present inventive technique is that hypoglycemia during the day is commonly associated with hypoglycemia at night. Hypoglycemia unawareness may occur in these situations and glycemic rebounds that produce large, frequently time altered (phase shifted) glucose excursions may occur. These excursions may also be recurrent if the hypoglycemia is recurrent. While individual glucose patterns may differ greatly in different patients, any given patient is likely to exhibit specific repetitive blood glucose level patterns on a daily cycle.

Non-repetitive glycemic patterns make effective glucose management difficult or impossible. However, many of the life events associated with non-repetitive glycemic patterns (such as food intake, work stress, emotional stress, exercise, pain, gastroparesis, hypoglycemia unawareness and changing sensitivity to insulin) are all potentially identifiable and quantifiable modifying factors. Furthermore, sleep, arousal, and menses, are examples of identifiable periodic factors that can alter glycemic patterns. Periodic factors affecting blood glucose levels may be circadian (occurring on a 24-hour basis), ultradian (occurring more frequently than every 24 hours) or infradian (occurring less frequently than every 24 hours).

Hypoglycemia unawareness in individual patients may result in recurrent hypoglycemia during sleep that is only apparent as hyperglycemia (glycemic rebound) during the morning. Over-treatment of this morning hyperglycemic episode may occur and result in a hypoglycemic episode during the afternoon that appears to be the first one of the day, but is actually the second. This is important because the first hypoglycemic excursion increases the risk of a second low glucose level due to increased sensitivity to insulin that occurs after the body responds to a hypoglycemic episode.

When employing conventional treatment strategies, these recurrent glycemic patterns typically result in a failure to achieve desired clinical diabetes management goals. Dyslycemic excursions resulting from such failures produce both acute and chronic diabetes complications. The present inventive technique predicts these unwanted glucose excursions and enables patients to prevent them from happening

The present inventive technique applies a new clinical strategy in combination with computer analysis to alter the clinical application of continuous glucose monitoring (e.g., by CGMS® or a similar system) from its usual form. Continuous glucose monitoring is performed in combination with maintenance of a life event diary that record significant events that can affect glucose levels and insulin sensitivity. Glucose monitoring data is analyzed in combination with the life event diary to create a patient-specific predictive model that permits development of a patient-specific treatment regimen that permits the patient to anticipate and prevent damaging dysglycemic excursions and provides critical information necessary to make effective adjustments to treatment plans. Effectively, the present inventive technique transforms diabetes management from being reactive to glucose levels that have already changed to a strategy that acts in anticipation of glucose levels that are about to change.

The present inventive technique collects data from individual patients and incorporates their glucose changing life events as well as periodic ultradian changes (occurring more frequently than once per day) in glucose insulin relationships while they sleep. It identifies and anticipates time-dependent changes in glucose levels and provides critical information needed to adjust insulin-glucose administration. The life event information is collected in a life event diary. It should be noted that this technique is distinct from, and supplemental to the continuous data collection functions provided by continuous glucose monitoring systems such as CGMS® produced by the Medtronic MiniMed Corporation.

The invention includes several elements. These are: a Life Event Diary system; a programmed system for personal computers, PDAs (Personal Digital Assistants), etc., to provide patients with diabetes mellitus a convenient, automated way of recording life event data in a suitable format for subsequent analysis; a continuous glucose monitoring system capable of recording blood glucose levels over an extended period of time; an analysis system (e.g., computer program) for analyzing ultradian life event data for the life event diary system in the context of data recorded by the continuous glucose monitoring system to produce a predictive mathematical model defining periods of vulnerability to unacceptable dysglycemic excursions (low and high) in the monitored patient and to produce a treatment strategy based upon that model; and an analysis system for identifying significant periods of dysglycemic excursion risk for a patient to physicians, technicians and other health professionals, or for use as a component in higher level systems.

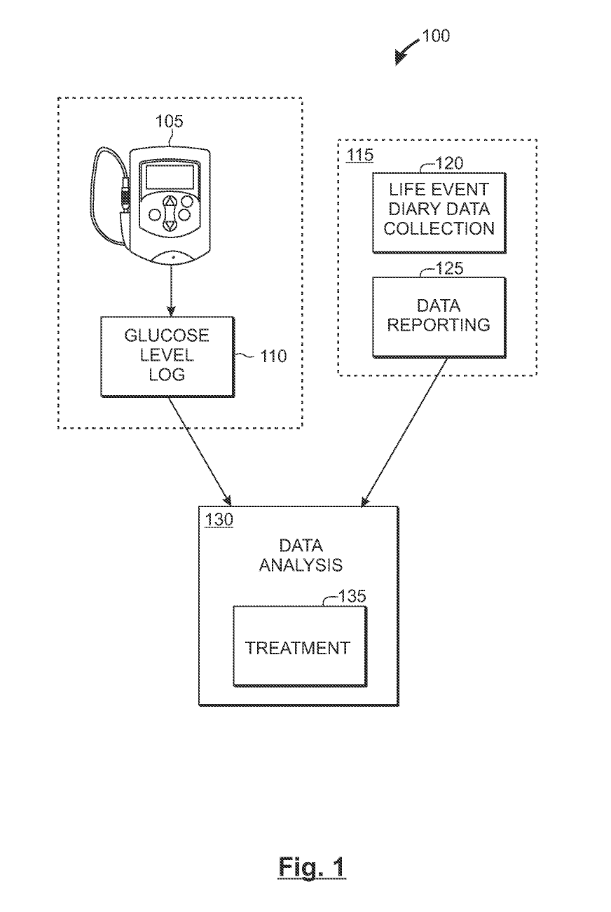

These elements are shown in FIG. 1. FIG. 1 is a block diagram 100 showing the various elements of the present inventive technique. In the Figure, a continuous monitoring system 105 is employed to record “continuous” blood glucose levels over an extended period of time into a glucose level log 110. A life-event diary system 115 comprising a data collection element 120 and a data reporting element 125 provides a means by which a diabetes patient can record significant life events for the time period during which continuous glucose monitoring is performed. An automated analysis system 130, typically implemented as one or more computer programs has a treatment element 135 for recognizing time-dependent glycemic patterns and developing a corresponding course of treatment and a reporting element for identifying significant periodic and non-periodic vulnerability to dysglycemic excursions.

The present inventive technique facilitates collection and formatting of clinically significant life event data and combining it with a “continuously” generated glucose data set, to highlight specific “time domains” of increased risk for dysglycemia in individuals with diabetes mellitus. By the mathematical transformation of these highlighted dysglycemic trends, the present invention provides an analytical technique that can be executed by a computer program to anticipate low and high glucose levels in diabetic patients when high-risk behaviors occur. These high-risk behaviors will generate alerts and alarms for individual patients based upon their ultradian glycemic trends.

Research into blood glucose levels associated with diabetes mellitus has shown that predictable patterns of glucose levels occur in selected situations in diabetic patients. These glucose trends are powerful hints to improved glucose management in these patients. Application of the Life Event Diary System and “continuous” glucose testing makes it possible to identify time-dependent and behavior-dependent glucose trends that represent recurrent ultradian physiologic changes in individual patients. Sleep onset and hypoglycemia change glucose trends and deprivation of sleep or avoidance of hypoglycemia alter insulin requirements in diabetic individuals. Analysis of glucose data sets generated by “continuous” glucose monitoring systems such as CGMS® provides greatly improved clinically significant information performed in the context of a life-event diary that permits clinical correlation of the glucose level data with life events known to have and effect on glucose levels and insulin sensitivity.

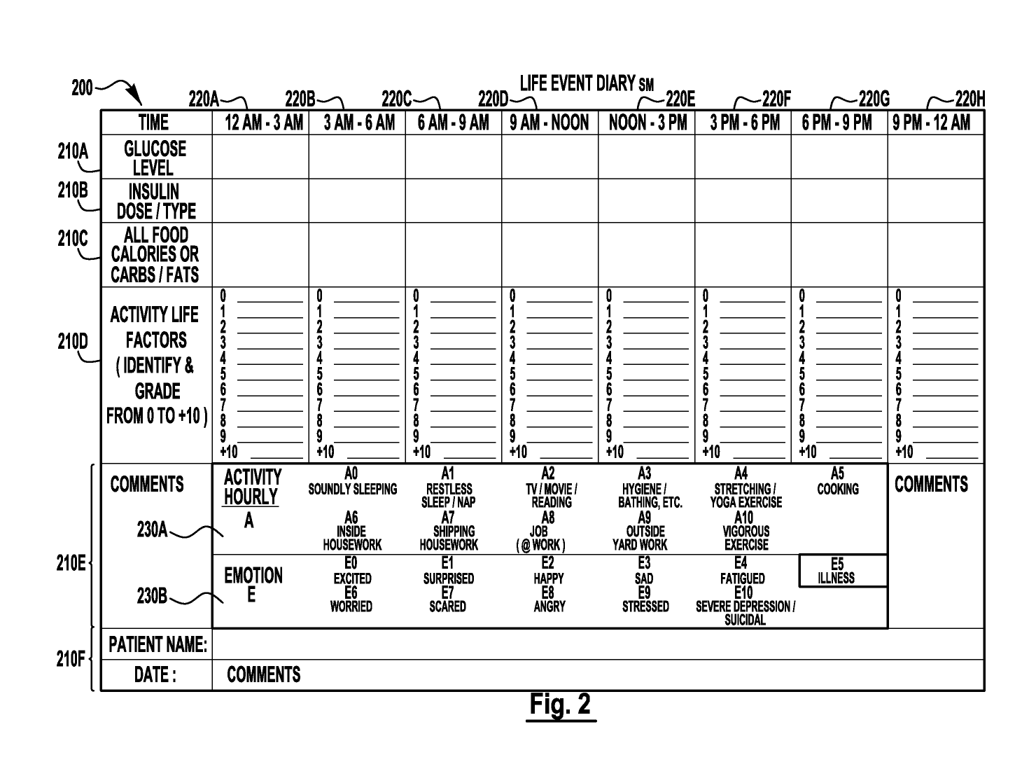

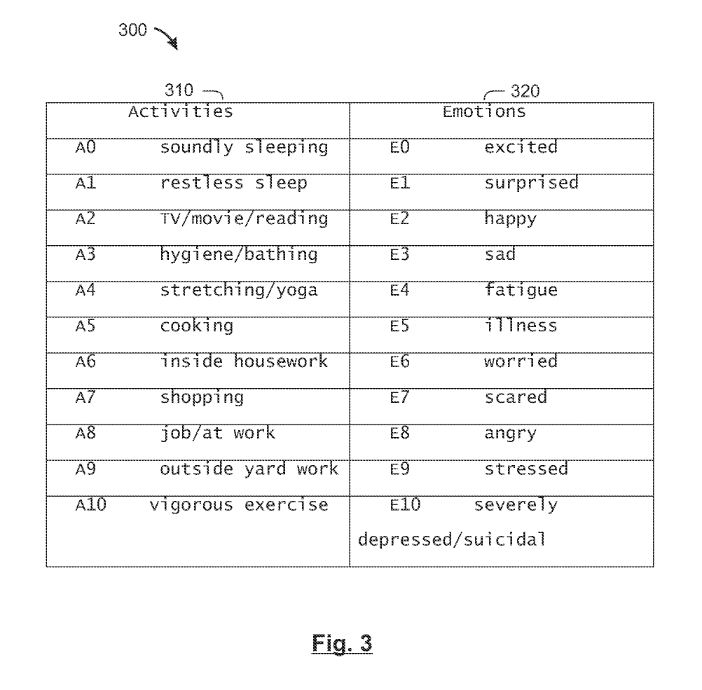

In order for the inventive system to model a patient's glycemic patterns accurately, it is essential that all relevant life events that occur during the period of continuous glucose monitoring are faithfully and accurately recorded in the life event diary. In order to help ensure that a patient is capable of keeping an accurate record of such life events, a 1-week training period is typically used to acquaint patients with the Life Event Diary System. Patients must be able to maintain this intensive diary format during “continuous” glucose monitoring such as CGMS® for the inventive technique to be applied successfully. The 1-week diaries are assessed for adequacy before the patients are permitted to proceed with further application of the inventive technique. Life event information recorded in the diary includes food intake (type an amount), insulin dosage (type and dose), hypoglycemia, and an alpha numeric grading of both activity/sleep and feelings/emotions. These are described in greater detail hereinbelow with respect to FIGS. 2 and 3.

FIG. 2 is a representative view of a life event diary form 200 for a single day of monitoring. As shown in the Figure, a patient would fill out the form 200 to record significant life events during continuous glucose monitoring. The information on the form 200, however, is ultimately entered into a processing system (e.g., computer or PDA) for subsequent analysis. The form 200 is organized generally into rows and columns. A glucose level row 210A is provided for the patient to record average glucose level readings for a plurality of time periods in the day. An insulin dose row 210B is provided for the patient to record the type and dosage of all insulin administered. A food row 210C is provided for the patient to record details of food intake. An activity row 210D is provided for the patient to record significant life events and factors (activities and emotional states) that can affect glucose levels. A “key” row 210E contains reference information 230A related to activity levels and emotional states 230B for the patient to refer to while filling in the activity row 210D. An information portion 210F of the form 200 is provided for recording the date and the patient's name. Eight columns 220A, 220B, 220C, 220D, 220E, 220F, 220G and 220H divide the glucose, insulin, food and activity rows 210A-D of the form 200 horizontally into eight equal 3-hour time periods covering one full day.

Typically, one form 200 would be filled out by the patient for each full or partial day of continuous monitoring. The information recorded by the patient is then used in combination with the continuous monitoring data to help identify trends in the patient's glucose level response to the activities, events, food intake, and insulin dosages recorded by the patient.

“Finger-stick” glucose levels are recorded for each of the eight equal 3-hour time periods on the in the glucose level row 210A of the form 200 over several successive days. The finger-stick levels can be used as a validity check against continuous monitoring. The type and dosage of any insulin administered during the eight equal time periods is recorded into the insulin row 210B. Similarly, any food intake for the eight equal 3-hour time periods is recorded into the food row 210C. The patient's activities and emotions are graded according to a quantitative scale as shown in FIG. 3 and recorded in the activity row 210D. Although shown and described in terms of a form 200, the process of life event data collection can readily be automated, e.g., via a program running on a personal computer, a PDA (personal digital assistant) or a pre-programmed calculator. Accordingly, the life event diary system 115 of FIG. 1 represents either a manual process of data gathering and transcription or an automated process carried out with the assistance of electronic hardware.

FIG. 3 is a table 300 of “quantitative” activity (exertion) and emotion levels. An activity level column 310 organizes and grades a variety of activities from the least amount of exertion A0 (soundly sleeping) to the greatest amount of exertion A10 (vigorous exercise). Although not necessarily a linear grading scale, the activities corresponding to the grade levels A0-A10 generally represent an increasing scale. That is, watching TV (A2) typically requires less exertion than bathing (A3) and cooking (A5) generally requires less exertion than housework (A6). Similarly, an emotions column 320 organizes and grades selected emotional states from most “upbeat” (E0—excited) to most despondent (E10—severely depressed/suicidal). As with the activity scale (A0-A10), the emotions scale (E0-E 10) moves in a generally monotonic fashion from one end of the scale to the other.

The patient uses the activity scale (A0-A10) and emotion scale (E0-E10) in FIG. 3 to help quantify his/her level of exertion and emotional state for the time period being recorded. Although questions like “On a scale of 0-10, with 0 being extremely happy and 10 being extremely sad, how happy or sad are you right now?” might get a reasonable response, the activity level scale and emotions scale in the table 300 help the patient to identify finer “shades” of exertion and emotion so that he/she can respond more consistently.

Blood glucose data from a continuous glucose monitoring system such as CGMS® can be retrieved in computer-readable format (e.g., by a program such as “MiniMed Graphs” for CGMS®). Blood glucose level readings are taken from a suitable glucose level sensor (e.g., Medtronic Mini Med CGMS® Glucose Sensor®) at a relatively high sample rate (e.g., every 5 minutes) and are recorded over an extended period of time (e.g., several days).

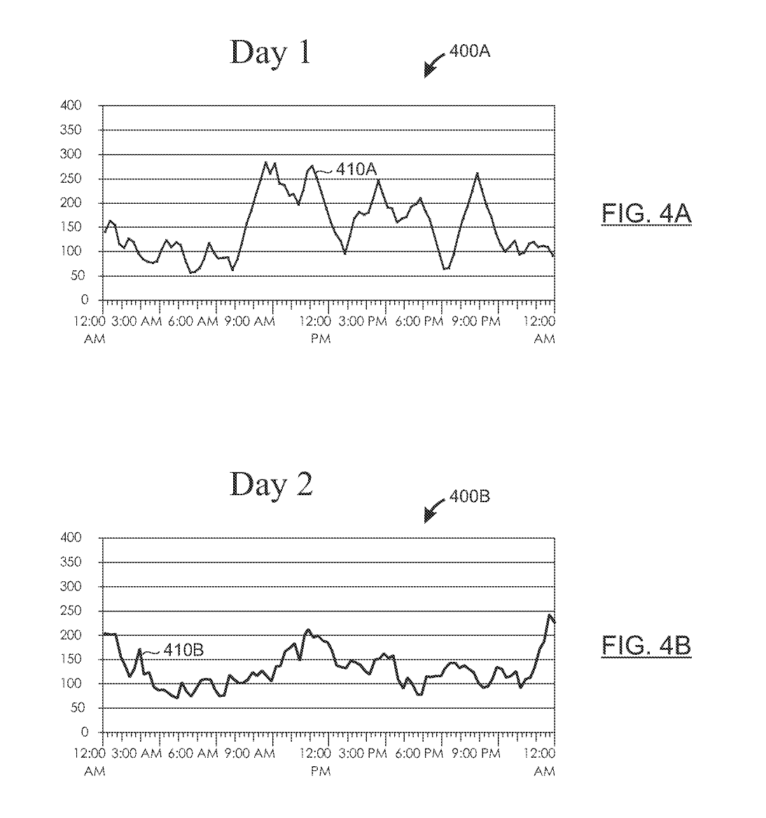

FIGS. 4A and 4B are graphs 400A and 400B, respectively, of “continuously” monitored blood glucose level readings for a particular patient on two successive days (“Day 1” and “Day 2”). The graph 400A of FIG. 4A shows a graph line 410A of blood glucose level (vertical axis) plotted against time (horizontal axis) for the first day of monitoring (“Day 1”). The graph 400B of FIG. 4B shows a graph line 410B of blood glucose level (vertical axis) plotted against time (horizontal axis) for the second day of monitoring (“Day 2”).

According to the present inventive technique, the blood glucose level readings from the continuous glucose monitoring system is then analyzed to identify hypoglycemic episodes and recurrent and non-recurrent (periodic and non-periodic) patterns of hypoglycemia and glycemic rebounds (that typically occur after hypoglycemia). A hypoglycemic episode is defined as existing during any time period where the blood glucose level is less than 70 mg/dl.

The continuous glucose data is also analyzed to identify correlations between events in the patient's life event diary (e.g., activity level, emotional states, insulin dosage, food intake) and glycemic fluctuations over the duration of continuous monitoring.

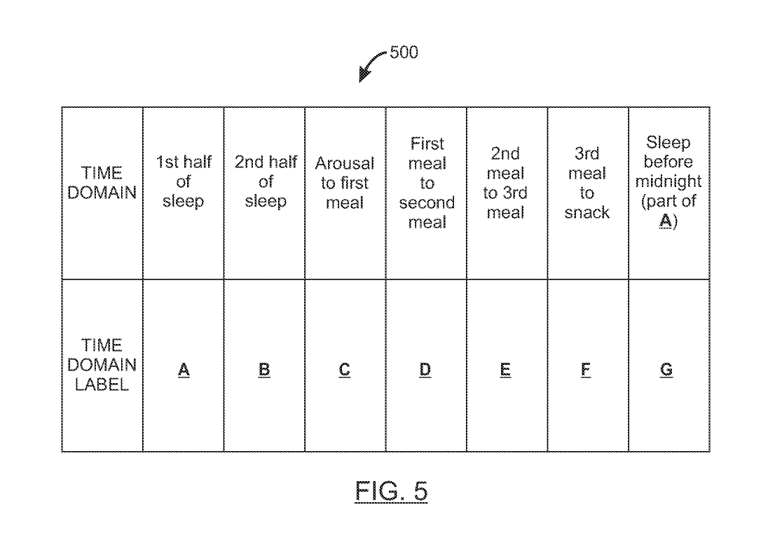

After initial analysis to identify gross glucemic periodicities and correlations of glucose level trends with life events as recorded in the life events diary, the resultant data is sorted into time domains based upon the timing of hypoglycemic episodes. FIG. 5 is a table 500 illustrating the organization of these time domains. In the table, a nominal “day” is broken up into seven time domains labeled “A”, “B”, “C”, “D”, “E”, “F” and “G”. Time domain “A” is the first half of the patient's sleep period. Time domain “B” is the second half of the patient's sleep period. Time domain “C” is the time between when the patient wakes up and the patient's first meal of the day. Time domain “D” is the time between the patient's first and second meals. Time domain “E” is the time between the patient's second and third meals. Time domain “F” is the time between the patient's third meal and a snack. Time domain “G” is the portion of the patient's sleep period that occurs before midnight (which is generally a part of time domain “A” for the next “day”). The time domains are determined based upon the information recorded in the patient's life event diary for the monitored period.

After defining the time domains, life events are organized into the time domains in which they occur. The continuous glucose monitoring data is then analyzed to identify hypoglycemic episodes by time domains to identify likely antecedent life events such as exercise and meals, as well as the relationship to periodic life events such as sleep. Factors to be identified include duration of hypoglycemic episodes and any subsequent hyperglycemic rebounds, repetitive episodes of hypoglycemic and hyperglycemic excursions, and the “area” of each such glycemic episode. The “area” of a glycemic episode is measured as a function of time against nominal baseline “normal” glucose levels from the beginning of the episode (excursion outside of normal levels) to the end of the episode (return to normal levels). Accordingly, a hyperglycemic episode would have a positive area above the “normal” levels and a hypoglycemic episode would have a negative area. The area has units of glucose level multiplied by time, e.g., mg-secs per dl.

To quantify glycemic episodes associated with specific life events, mathematical modeling of glucose levels is performed to achieve a best fit to a sine wave. Waveform analysis of glucose levels is performed on patient data to convert glucose level variations related to recurrent glycemic excursions and pattern altering life-events into a more mathematically usable sine wave model format. The patterns resulting from this computerized transformation are then analyzed for their degree of conformation (correlation with) the recorded data.

Mathematical Analysis is Performed in 2 Stages:

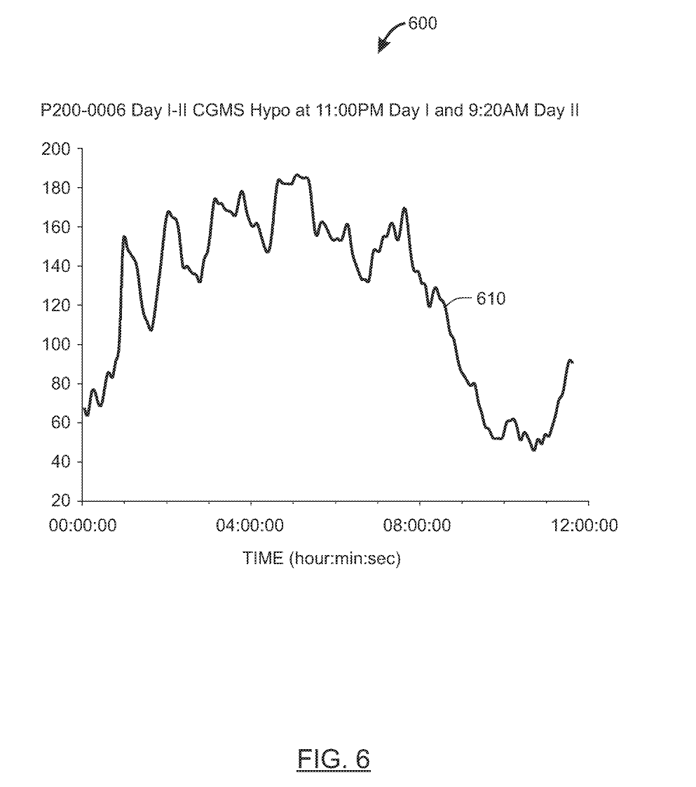

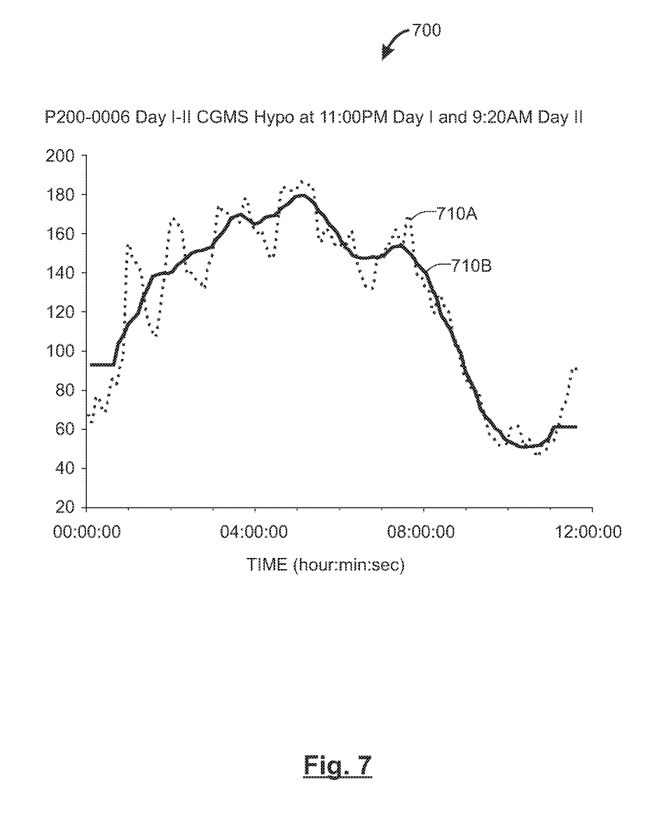

Using a software rolling average algorithm (or any other suitable averaging/smoothing technique), the glucose levels obtained from a “continuous” glucose monitoring such as CGMS® the glucose patterns are “smoothed” to de-emphasize erratic glucose level variations that may have occurred during the data acquisition. The erratic glucose level variations represent measurement noise, including normal glucose sensor variability and monitoring artifacts. By smoothing or “filtering” these highly erratic components of the monitored glucose waveform, overall glucose level trends related to the patient's life events become easier to identify. This is shown and described with respect to FIGS. 6 and 7.

FIG. 6 is a graph 600 of continuously monitored glucose levels over a 12-hour interval. A graph line 610 plots the “raw”, unfiltered (un-smoothed) glucose levels (vertical axis) against time (horizontal axis). The graph line 610 exhibits considerable “jaggedness” from a variety of sources, including measurement noise and variability.

FIG. 7 is a graph 700 contrasting the “raw” glucose levels 710A (compare 610) with a graph line 710B representing the same glucose level data after smoothing as described hereinabove. Note that in the smooth data, most of the “jaggedness” is eliminated, resulting in a smooth trend line.

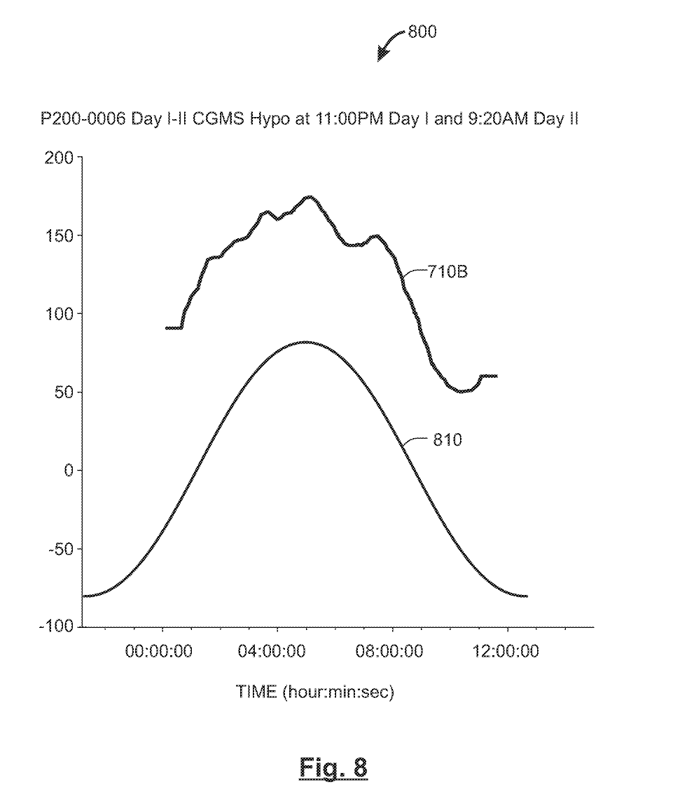

After smoothing, a Fourier analysis (Fourier transformation) is used to determine frequency components of the smoothed curve. A commercially available program such as SigmaPlot 8.® can be used to do both the smoothing and Fourier analysis. Fourier analysis expresses a time-domain waveform (e.g., the smoothed glucose level curve) as a corresponding frequency domain curve wherein each point along the frequency curve represents a sinusoidal component (sine wave) with a specific amplitude, frequency and phase. A fundamental component is identified (typically the lowest significant peak amplitude at a nonzero frequency the Fourier frequency curve) and its phase and amplitude are determined. The corresponding time domain sine wave is shown plotted against the smoothed data in FIG. 8.

FIG. 8 is a graph 800 of the smoothed glucose curve 710B (compare FIG. 7) against a corresponding sine wave curve 810 determined by Fourier analysis, as described hereinabove. Note that the sine wave curve 810 has its peak at essentially the same time (along the horizontal axis) as the smoothed glucose level curve 710B, and has generally the same shape and the same vertical scale (i.e., the glucose level curve 710B, if laid directly over the sine wave curve 810, would conform well thereto).

For the present inventive technique to accurately predict glycemic excursions, the sine wave resulting from analysis of any given time period must be highly representative of the glucose level waveform. That is, there must be a high degree of correlation between the sine wave and the glucose level curve. Sinusoidal patterns between recurrent hypoglycemic events are analyzed for duration of hypoglycemia and subsequent rebounds as well as for the area associated with positive (hyperglycemic) and negative (hypoglycemic) glucose level excursions represented (predicted) by the sine wave.

The present invention employs CAPR, or Computer Assisted Pattern Recognition to model patterns of glycemic variation identified by analyzing the life event diary in the context of continuous glucose monitoring. For purposes of the present invention, CAPR is a mathematical modeling technique by which ultradian patterns in glucose levels can be identified and approximated as closely as possible by sinusoidal functions. Ultradian patterning of glucose levels is grouped into categories based on identification of correlations between specific life events and glycemic excursions. Many patients who experience hypoglycemia at night also experience subsequent hypoglycemic trends the following early afternoon, and patients who experience hypoglycemia in the early afternoon experience subsequent hypoglycemic trends at night during sleep. These occurrences typify the sinusoidal pattern of recurrent glycemic trends (e.g., hypoglycemia), as illustrated by the sinusoidal model waveform 810 of FIG. 8.

Statistical analysis of glycemic trends shows that diabetic patients have two major types of glycemic patterning: 1) afternoon and subsequent night (AN), and 2) night and subsequent afternoon (NA). This applies especially to hypoglycemia and depends on the timing of the initial hypoglycemic or other recurrent glycemic excursion.

As described hereinabove, the present inventive technique accepts life-event data, continuous glucose monitoring data, etc., in computer-readable form and analyzes this data for periodicities in glucose level variations. These periodicities can be related to daily cycles, or to pattern altering events recorded in the life-event diary. These periodicities are modeled as sinusoidal waveforms which are used to predict glycemic excursions based upon observations of glycemic responses during monitoring.

To better understand the mechanism by which the modeling is accomplished, it is necessary to consider a sinusoidal function G(x) that has a pattern and a phase (e.g., G(x)=a sin(kπ(x−x0)/b) where ‘a’ is the amplitude of the sine wave, b is its period (in units of x—time, for purposes of the present invention), k is a frequency scale factor, and x0 is a reference point in the domain of x. This sine wave function provides a simple mathematical model to characterize hypoglycemic or other dysglycemic periodicity in diabetic patients. For example, the period of a sine wave can be determined as a full period of oscillation when:

$${\int }_{0}^{T} G ( x ) = 0$$

where T is the period and can be determined via evaluation of the integral. That is, the integral of a sinusoid is zero when integrated over any exact multiple of one cycle of the sinusoid, regardless of phase of the sinusoid with respect to the period of integration. The function G(x) models periods of increased dysglycemic risk by applying observed times and time intervals of recurrent dysglycemic excursions in individual patients during monitoring.

The CAPR technique of the present invention is illustrated in the steps of the following example analysis:

Step I: Determine parameters of G(x): Two different constants for b (period of the sine wave) are used, depending on the glycemic patterning. A ‘b’ value of 0.2042 is used for diabetic patients exhibiting AN patterning, and a ‘b’ value of 0.1325 is used for patients exhibiting NA patterning. The constant ‘a’ is referred to as the “euglycemic threshold” for all patients, in this example a=126 mg/dl. The value for x₀ is a reference position whose value depends upon the specific software smoothing and Fourier analysis techniques being used. It represents the leftmost point on the graph of FIG. 8 and determines the phase of the sinusoidal waveform. In this example case, x0 is generated by SigmaPlot 8 computer software as a byproduct of analysis.

The euglycemic threshold “a” of 126 mg/dl used for purposes of this example represents a fairly conservative diagnostic threshold blood glucose level for Diabetes. However, this threshold level may be too low in many practical applications of the present inventive technique, especially since acceptable post meal threshold levels can rise as high as 180 mg/dL. A higher, compromise euglycemic threshold (e.g., 150 mg/dl) can be substituted for wider applicability. The selection of the euglycemic threshold level depends largely upon therapeutic goals. For example, when treating a patient with hypoglycemia unawareness, a higher number (allowing for greater “swing” in glucose levels) is more appropriate to avoid excessive “false alarms” at lower levels. On the other hand, when treating a critically ill patient whose glucose levels may easily run high, then a lower euglycemic threshold (e.g., 126 mg/dL) is appropriate.

Step II: From each diabetic patient's “continuous” glucose monitoring data, the aforementioned CAPR technique (i.e., smoothing, Fourier analysis) is used to generate a characteristic sine wave pattern. Constants a and b are substituted into the sine wave analysis and the value of x₀ generated by the SigmaPlot 8 computer software is used.

Step III: Values xₙ are determined for each patient for hypoglycemia at times x when G(x) is equal to a suitable hypoglycemia threshold level, for example G(x)=70 mg/dl. These xₙ values x represent the start and end times ‘x’ of a hypoglycemic excursion. The start point occurs when G(x) crosses downward through the threshold level. The endpoint occurs when G(x) crossed back upward through the threshold. For patients exhibiting AN (afternoon-night) patterning xₙ=0.130 (k %-%). For patients exhibiting NA (night-afternoon) patterning xₙ=0.084 (k %-%). To maintain a 24-hour scale it is necessary to use values of 1 and 2 for k; using other positive integers results in values that diverge from a 24-hour scale. To determine the 24-hour value it is necessary to subtract multiples of 24 hours until the desired result is acquired. Typically, the value of % is zero; that is, % is not typically used. However, it is included in the equation as a reminder that the sinusoidal response can be skewed by a variety of factors. For example, the failure of different vital organs can have an effect on glycemic modulation.

Values of x can be converted to hours via the following conversion:

$$G ( x ) = a sin ( k - \text{?} )$$

$$\text{?} \text{indicates text missing or illegible when filed}$$

where: a is a predetermined “euglycemic” threshold (e.g., 150 mg/dl), b=constant (typically 0.1325 for NA glycemic patterning/constant 0.2042 for AN glycemic patterning), x0=−0.0516728.

Predicted times of hypoglycemic events are calculated by determining the times when G(x) crosses 60 and 80 mg/dl threshold levels (yielding two results per threshold level—the downward crossing times (start) and the upward crossing times (end). Similarly, predicted times of hyperglycemic events are calculated by determining the times when G(x) crosses 180 and 200 mg/dl threshold levels (yielding two results per threshold level—the upward crossing times (start) and the downward crossing times (end).

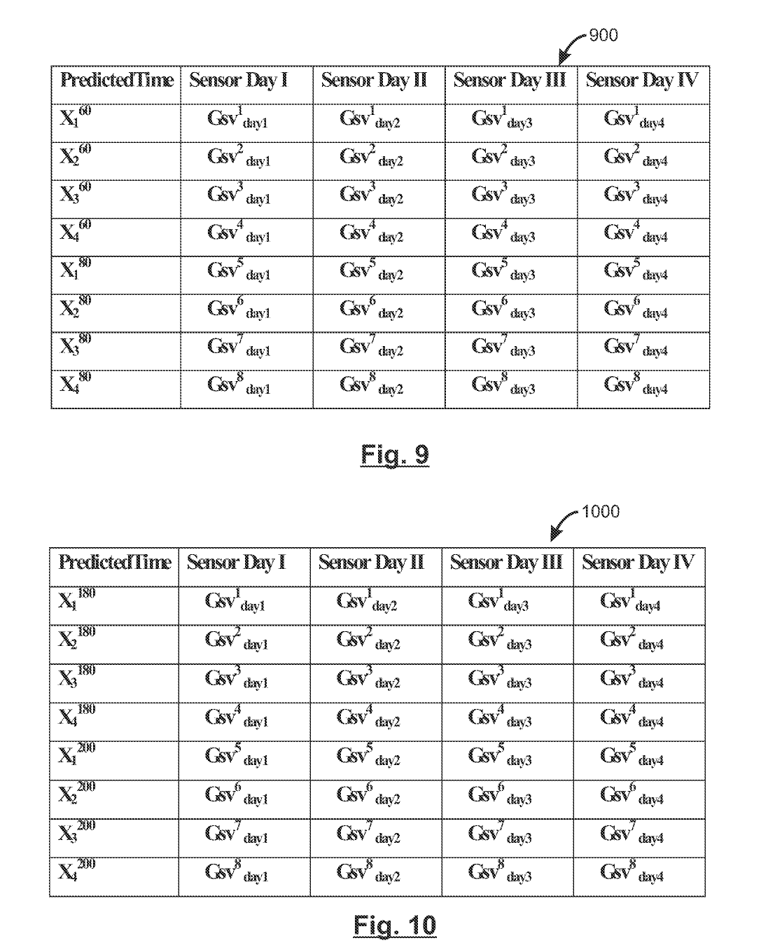

Step IV: Each value of x corresponds to a time or time interval of dysglycemic susceptibility. When values are converted to hours it becomes possible identify the timing of hypoglycemic vulnerability on any given day as is shown in the table 900 of FIG. 9, while that for hyperglycemic vulnerability is shown in the table 1000 of FIG. 10. In the tables of FIGS. 9 and 10, the “x” values in the leftmost column refer to

In FIGS. 9 and 10, the subscripts of “x” are ordinals that indicate specific reference times. The superscripts of “x” indicate the threshold level crossed at time “x”. That is, x₁¹⁸⁰ refers to a first time ‘x’ when glucose levels are predicted to cross a 180 mg/dl threshold level. Similarly, x₂¹⁸⁰ refers to a second time ‘x’ when glucose levels are predicted to cross the 180 mg/dl threshold level. The time interval between x₁¹⁸⁰ and x₂¹⁸⁰ represents a time window of glycemic vulnerability. These times xₙ are determined by Fourier modeling of the continuously monitored glucose levels, as described hereinabove. The values G_{SV}ⁿ represent the measured glucose levels at time ‘n’ for each day of continuous monitoring.

By anticipating times of dysglycemic vulnerability for individual diabetic patients in the manner described above with respect to the present inventive technique, it is possible to control glucose intake and insulin administration in anticipation of a predicted event to prevent the event from occurring, or to lessen its depth. Since glycemic rebounds often result in overmedication and since dysglycemic excursion can be extremely damaging, using prediction to provide treatment that prevents large dysglycemic excursions both minimizes damage and makes maintenance of clinical goals easier and more reliable.

The Life Event Diary System (in software) provides a simple, automated way of assisting the patient in recording complete and accurate life event information during continuous glucose monitoring. This further enables the physician (manually) or a data analysis system (automatically) to identify times of dysglycemic vulnerability. By identifying those critical times of vulnerability, an appropriate course of treatment can be devised that anticipates those vulnerabilities and “gets ahead of them” to prevent dysglycemic excursions.

Clinical application of the present inventive technique (i.e., computerized intensive life event diary programs with mathematical modeling of continuously generated glucose data) anticipates and alerts patients with diabetes mellitus to increased vulnerability to low and high glucose levels. This changes the paradigm of treatment in people with diabetes mellitus by making insulin delivery prospective (in advance of anticipated events) based on the individual's life events and physiologic responsiveness, instead of being generalized or reactive to infrequently measured high or low glucose levels that have already occurred, as is typical of current treatment of diabetes mellitus.

This system has the potential to greatly improve glucose control over time, thereby improving quality of life and clinical outcomes by avoiding acute and chronic complications of diabetes. Application of the present invention is specifically intended to avoid the major impediment to effective diabetes control, namely hypoglycemia. By preventing hypoglycemia, the present invention will also prevent adverse effects from hypoglycemia unawareness as well as rebound hyperglycemia. The present inventive technique can recognize glycemic effects of exercise, sleep, or work in individuals

This present inventive technique is designed to prevent hyperglycemia as well. It will recognize meals or mealtimes associated with inadequate insulin use, as well as life events that require increased insulin doses such as emotional stress, pain, menses or arousal.

Using a transformed sign wave function of glucose level variations in DMI (diabetes mellitus type I) patients to represent periods of dysglycemic vulnerability, it is possible to predict recurrent hypoglycemia or hyperglycemia in individual patients. Using this approach, an alarm system can be employed based upon modeled values to warn patients of impending glycemic excursion, enabling them to adjust their insulin usage or food to prevent hypoglycemia or hyperglycemia.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

What is claimed is:

1. (canceled)

2. A method according to claim 21, further comprising:
determining anticipated times when the patient's blood glucose levels will cross hypoglycemic and hyperglycemic threshold crossings, based upon times when said sinusoidal function crossed said threshold.

3. A method according to claim 2, further comprising:
defining windows of glycemic vulnerability during periods of time between said anticipated times.

4. A method according to claim 2, further comprising:
determining an appropriate plan of treatment based upon said anticipated times such that treatment is administered in anticipation of predicted dysglycemic episodes.

5. A method according to claim 2, further comprising:
correlating recorded life-event information with corresponding fluctuations in recorded glucose levels to determine specific glycemic responses to specific life events.

6. A method according to claim 21, further comprising:
determining said predictive sinusoidal function by Fourier analysis of recorded continuous glucose monitoring data.

7. A method according to claim 21, further comprising:
recording said life-event information in a life-event diary in electronic form by means of a computing device.

8. A method according to claim 7, wherein:
said computing device is a computer.

9. A method according to claim 8, wherein:
said computing device is a PDA (personal digital assistant).

10. A system for predicting dysglycemic excursions in diabetes mellitus patients, comprising:
a continuous glucose monitoring system for recording a patient's glucose levels over an extended period of time;
a life-event diary system for recording life-event information during continuous glucose monitoring; and
means for analyzing recorded glucose level information in the context of life-event information recorded by the life-event diary system to determine a model sinusoidal function that closely approximates glucose levels observed during monitoring; and
means monitoring and recording the patient's blood glucose levels repeatedly over short intervals of time for an extended period of time of at least 72 hours to provide continuously generated glucose data with a continuous glucose monitoring system;

11. A system according to claim 10, further comprising:
means for determining anticipated glucose threshold crossing times by determining times when said model sinusoidal function crosses those threshold levels.

12. A system according to claim 11, wherein:
periods of time between said anticipated times define time windows of glycemic vulnerability.

13. A system according to claim 12, further comprising:
means for correlating recorded life-event information with corresponding fluctuations in recorded glucose levels to determine specific glycemic responses to specific life events.

14. A System according to claim 12, further comprising:
means for performing Fourier analysis of recorded continuous glucose monitoring data to determine said model sinusoidal function.

15. A system according to claim 2, wherein:
said life-event diary system further comprises a computing device for recording said life-event information in a life-event diary in electronic form.

16. A system according to claim 6, wherein:
said computing device is a computer.

17. A system according to claim 16, wherein:
said computing device is a PDA (personal digital assistant).

18. A system for predicting dysglycemic excursions in diabetes mellitus patients, comprising:
a continuous glucose monitoring system for recording a patient's glucose levels over an extended period of time;
a computing device for recording life-event information during continuous glucose monitoring;
computing means for analyzing recorded glucose level information in the context of life-event information recorded by the life-event diary system to determine a model sinusoidal function that closely approximates glucose levels observed during monitoring; and
computing means for determining anticipated glucose threshold crossing times by determining times when said model sinusoidal function crosses those threshold levels.

19. A system according to claim 18, further comprising:
means for correlating recorded life-event information with corresponding fluctuations in recorded glucose levels to determine specific glycemic responses to specific life events.

20. A method for predicting time-dependent glycemic patterns in a diabetes mellitus patient, comprising:
monitoring and recording the patient's blood glucose levels repeatedly over short intervals of time for an extended period of time of at least 72 hours to provide continuously generated glucose data with a continuous glucose monitoring system;
recording life-event data about the patient, including food intake, work stress, emotional stress, exercise, pain, gastroparesis, and sleep, arousal, and menses, for the extended period of time over which the patient's blood glucose levels are repeatedly monitored with a programmed system for recording the life-event data;
analyzing the life-event data occurring more frequently than every 24 hours in the context of the continuously generated glucose data with the automated analysis system to define periods of vulnerability to unacceptable dysglycemic excursions of the patient when high risk life-events occur; and
identifying significant periods of dysglycemic excursion risk for the patient based upon anticipated unacceptable dysglycemic excursions of the patient based on the analysis of the identified correlations between the life-event data occurring more frequently than every 24 hours in the context of the continuously generated glucose data.

21. The method according to claim 20 further including determining a predictive sinusoidal function from analyzing life-event data more frequently than every 24 hours to closely match periodic variations of blood glucose levels
